(19) United States
(12) Patent Application Publication
(10) Pub. No.: US 2020/0086070 A1
(43) Pub. Date: Mar. 19, 2020

(54) NEBULIZER UNIT WITH EXCHANGABLE MOUTHPIECE

(71) Applicant: NEBU-TEC MED. PRODUKTE EIKE KERN GMBH, Elsenfeld (DE)

(72) Inventor: Stefan Kern, Elsenfeld (DE)

(21) Appl. No.: 16/317,140

(22) PCT Filed: Jul. 13, 2017
(86) PCT No.: PCT/DE2017/100582

§ 371 Date: May 17, 2019

(30) Foreign Application Priority Data
Jul. 14, 2016 (DE) 10 2016 112 990.9

(51) Int. Cl. A61M 15/00; A61M 11/00

(52) U.S. Cl. CPC A61M 15/0021; A61M 11/005

(57) ABSTRACT

A nebulizer unit includes a housing, an aerosol generator, and an exchangeable mouthpiece. The aerosol generator is supported within the housing by a sealing ring and a retaining structure, and includes an outlet end having an integrally formed mouthpiece connector. The exchangeable mouthpiece is configured to be received by the mouthpiece connector. One side of the mouthpiece includes at least one air channel, each air channel having a first end that opens into an aerosol chamber. The aerosol chamber is delimited at an end face by the aerosol generator and circumferentially by the mouthpiece connector and the mouthpiece.

### CROSS-REFERENCE TO RELATED APPLICATION

This application is a Section 371 National Stage Application of International Application No. PCT/DE2017/100582, filed Jul. 13, 2017, the content of which is incorporated herein by reference in its entirety, and published as WO 2018/010734 on Jan. 18, 2018, not in English.

### FIELD

Embodiments of the present disclosure relate to a nebulizer unit having an exchangeable mouthpiece.

### BACKGROUND

For treating diseases of the respiratory tract, inhalation therapy is often recommended, in which a medication solution is atomized to form an aerosol comprising fine droplets that are micrometers in diameter. The advantage of administration in this form is that the medication is taken directly to the place of action, which, on one hand, is faster and more efficient than transport through the bloodstream.

For a nebulizer used in inhalation therapy, a nebulizer unit with mouthpiece usually comprises a medication reservoir and a control unit, which is connected to the nebulizer unit and controls the aerosol generator which is present in the nebulizer unit. The aerosol generator, in the case of modern nebulizers, comprises a metal mesh membrane and a vibration generator, as well as, possibly, a mechanical coupling portion in the form of a carrier plate. The mesh membrane is a thin metal plate, which, in a central region, comprises fine, that is to say micrometer-sized, funnel-shaped pores, the larger cross-section of the funnel being directed towards the reservoir. The vibration generator is in general a circular piezo-crystal, which is mechanically coupled to the mesh membrane, possibly by means of a carrier plate connected between, and enclosing the perforated region. Electrical excitation and the resulting deformation of the piezo-crystal effects a bending of the perforated region of the mesh membrane, which vibrates in a frequency determined by the electrical excitation signal, forcing a medication fluid at a feed end into the fine pores and emitting it at the outlet end in the form of an aerosol of micrometer-fine drops.

So that a user can also use this nebulizer unit, a mouthpiece must be integrally formed or mounted, of which the front end is designed such that it can be easily enclosed by the lips, so that an airtight, or largely airtight, closure can be produced. It is thus made possible for the user, when inhaling, to suck in air through the volume present at the outlet end of the aerosol generator, within the nebulizer unit and the mouthpiece, which mixes with the aerosol generated there and ideally passes into the bronchial tubes or the lung. In order that the removal can function, air inlet holes must be present in the volume present at the outlet end of the aerosol generator. In the nebulizers of the prior art, they are usually present in the nebulizers unit itself.

The disadvantage of this is that, on one hand, cleaning is made difficult thereby and, on the other hand, the air quantity drawn in or the pressure needed to suck in the required air quantity cannot be easily adjusted. The latter is important for the inhalation convenience of the user.

### SUMMARY

Embodiments of the present disclosure attempt to overcome the above-described disadvantages and has the object of providing a nebulizer unit with an exchangeable mouthpiece that permits simple cleaning and a simple adaptation of the air flow to a degree desired by the user or appropriate for the therapy.

This object is solved by means of a nebulizer unit having an exchangeable mouthpiece. The mouthpiece is designed so as to be exchangeable, that is to say is formed with one end such that it can be connected to or mounted on a mouthpiece connector or connection means on the nebulizer unit. In some embodiments, the nebulizer unit includes a cylindrical nozzle at an aerosol-generator outlet end, on which a complementary, rear portion of the mouthpiece can be plugged. A cylindrical shape is preferred since it can be most simply and expediently prepared, however, any other shape of the cross-section is also conceivable, such as an oval and/or rectangle and/or polygon, with or without rounded corners.

In some embodiments, the exchangeable mouthpiece of the nebulizer unit includes one or more air inlet channels that are integrally moulded on it. These are preferably located on one side of the mouthpiece, with one, the front opening of the channel or channels opening into a volume formed by the mouthpiece connecting piece of the nebulizer unit and by the mouthpiece itself, which is located on the outlet end of the aerosol generator. The other, rearward end of the air channels is open outwardly, so that air can thereby be sucked in. If a plurality of channels are present, the rear end of one of these channels can also be connected to a sensor or to a flow channel that is present on the outside of the nebulizer unit and leads to sensor.

The advantage of the exchangeability of the mouthpiece consists in the fact that, for each user, a suitable mouthpiece can already be kept ready, which is adapted to the needs of the user, both in the form of the part that is gripped by the lips and in the size, that is to say the clear width of the air channels. Another important advantage is the simplified cleaning of the aerosol generator according to embodiments of the present disclosure, in that the removable mouthpiece of the portion of nebulizer unit that is at the aerosol generator outlet end is more easily accessible and the mouthpiece itself, since it does not contain any sensitive parts, can be easily washed or sterilized. In that the air inlet channels are present in the mouthpiece itself, it is also achieved that in the case of possible exhalation through the mouthpiece and nebulizer, less condensate passes as far as the aerosol generator and contaminates the latter. This, too, contributes to increased hygiene of the nebulizer unit with exchangeable mouthpiece according to embodiments of the present disclosure.

The quantity of sucked-in air can advantageously be more easily adjusted in that mouthpieces with differently sized air channels can be used. If more air is to be sucked in, for example in order to dilute the aerosol more greatly and not cause an irritation of the user's throat, a mouthpiece with larger openings can be easily mounted. In the case of nebulizer units that have an opening in the unit itself, a change of the sucked-in air quantity is not so easy. If at least one of the air channels of the nebulizer unit with a mouthpiece according to the embodiments of the present disclosure is designed such that it terminates with its rear end flush with one end of flow channel that is present on the nebulizer unit and leads to a pressure sensor. In that the effective take-off point of the pressure measurement in that portion of the aerosol chamber facing the mouthpiece is thereby shifted, a clearer signal can be measured and the breath profile of the user during inhalation can be more accurately monitored.

If a flush termination is to be produced, embodiments of the present disclosure may be configured such that either the cross-section of the connecting piece as well as the rear portion of the mouthpiece have no symmetry, that is to say are not cylindrical or essentially cylindrical, but, in the mouthpiece or in the connecting piece of the nebulizer unit, a groove as well as a notch that is complementary thereto in the other portion is present, so that the plugging together can only ever take place in one particular orientation, and an undesirable twisting, such that the air channel of the mouthpiece leading to the flow channel is not flush therewith, is precluded.

Advantageous embodiments of the present disclosure, which can be realized individually or in combination, in so far as they do not obviously preclude one another are described below.

In one embodiment, the mouthpiece of the nebulizer unit has a single air channel with the properties described above. Advantageously, however, further air channels may be present. In particular, it is advisable to provide three or more air channels, since, thereby, even when one of these air channels is used for connection to a pressure or flow measurement sensor, two open channels are still present for air suction. Ideally, these should be arranged such that good mixing of the sucked-in air with the generated aerosol is ensured and, as far as possible, a complete removal. On one hand, since, for medical reasons, as much of the medication as possible ought to reach its target, and on the other hand, there is also a hygienic advantage, since, due to the complete removal of the aerosol, it is prevented that the medication fluid in the aerosol chamber is deposited as a condensate at the outlet end of the aerosol generator and leads to an increased need for cleaning.

The mounting piece for the mouthpiece integrally moulded on the nebulizer unit can either be cylindrical or else have an oval, elliptical or polygonal cross-section. For reasons of simple manufacture, a cylindrical shape is preferred. In some embodiments an air channel is used on a flow channel of the nebulizer unit that leads to a pressure or flow sensor, mounting with an incorrect orientation is avoided by means of a notch, either in the connecting piece or on the mouthpiece, and a complementary groove is integrally formed in the other portion in each case.

In one embodiment, the mouthpiece of the nebulizer unit possesses more than one, and in particular precisely three air channels. Two are to thereby be placed in a symmetrical position on one side of the mouthpiece in each case and one on an underside. This number of air channels is therefore advisable, since even when a channel is used, for example located on the underside, as a feed line for a flow channel of the nebulizer unit two further are available, which, when symmetrically positioned, ensure good mixing of the sucked-in air with the aerosol produced in the aerosol chamber and ensure extensive removal of the diluted aerosol.

As a preferred material of the exchangeable mouthpiece, plastic is advisable, due to its favourable manufacture and low weight and the general chemical resistance, which facilitates cleaning and sterilization.

One embodiment of the air channel of the mouthpiece is outwardly open at a rear end, but can, however, also be connected such that it terminates flush on an inlet opening of a flow channel leading to the pressure sensor or flow sensor, which is, for example, integrally moulded on the underside of the housing of the nebulizer unit according to some embodiments. In the first case, the air channel fulfils its main intended purpose, namely to permit the sucking in of air from the outer side into the interior of the nebulizer unit and to ensure removal of the aerosol generated. Particularly preferably here, the air channels of the mouthpiece are designed such that their insides are in each case formed by the outside of the connecting piece of the nebulizer unit. When the mouthpiece is removed, each part of the mouthpiece is thus easily accessible for cleaning. The air channels, which are only open at both ends, then “arise” only when the mouthpiece is plugged onto the nebulizer unit according to some embodiments.

In a housing of the nebulizer unit, openings can either be present additionally, which permit a stronger air intake, or the aerosol chamber is designed such that it is only open at the side leading to the mouthpiece, so that air inlet is subsequently only possible via the air channels of the mouthpiece.

In some embodiments, the nebulizer unit includes a medication reservoir into which the medication liquid to be inhaled can be filled, and which can be closed firmly and liquid-tight by means of a cap.

Further details and features of embodiments of the present disclosure are described in greater detail below with reference to the figures of preferred embodiments. These are only intended to illustrate the embodiments, and in no way to limit them.

### BRIEF DESCRIPTION OF THE DRAWINGS

In detail,

FIG. 1 shows a perspective view of an embodiment of the exchangeable mouthpiece

FIG. 2 longitudinal section through an embodiment of the nebulizer unit with mounted exchangeable mouthpiece

### DETAILED DESCRIPTION OF ILLUSTRATIVE EMBODIMENTS

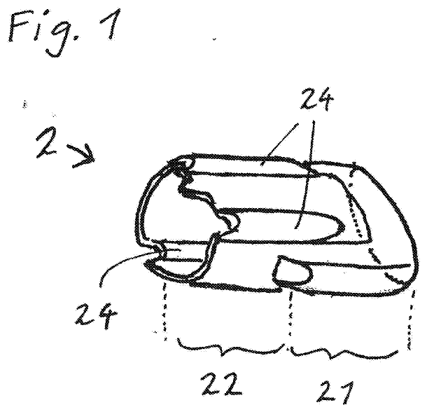

FIG. 1 shows, in perspective view, an exchangeable mouthpiece 2 in accordance with embodiments of the present disclosure. As can be seen, the mouthpiece 2 is essentially divided into two sections 21,22. The front on 21 has an oval cross-section and is formed and dimensioned such that a user can easily grip it with the lips and enclose it in an airtight manner. This front portion 21 merges continually into the rear portion 22, which serves for plugging the nebulizer unit 1 onto the connecting piece 12. This rear part 22 has essentially a circular cross-section, on which protrusions 24 with a semi-circular section, which are offset by approx. 120 degrees with respect to one another are mounted. They are completely open in the condition where they are not plugged and are therefore easily accessible for cleaning. If, however, the mouthpiece 2 is pushed or plugged onto the cylindrical connecting piece 12 of the nebulizer unit 1, air inlet channels 234 are formed by the outside of the connecting piece 12 and the protrusions 24. It is thus possible, to design the nebulizer unit 1 such that the aerosol chamber 120, present at the aerosol generator outlet end, is only open at the end face leading to the mouthpiece and otherwise is completely closed, that is to say that no other air-inlet openings are present.

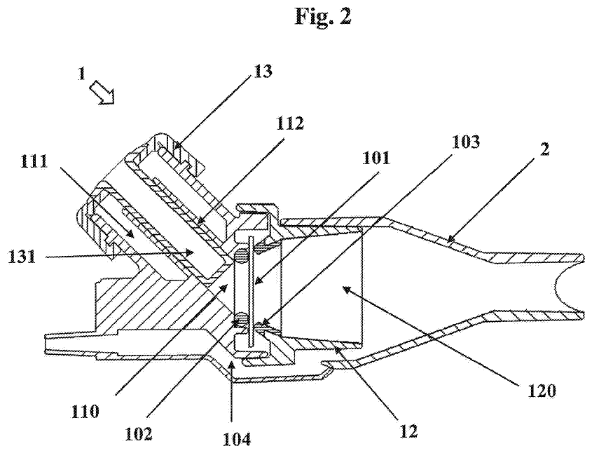

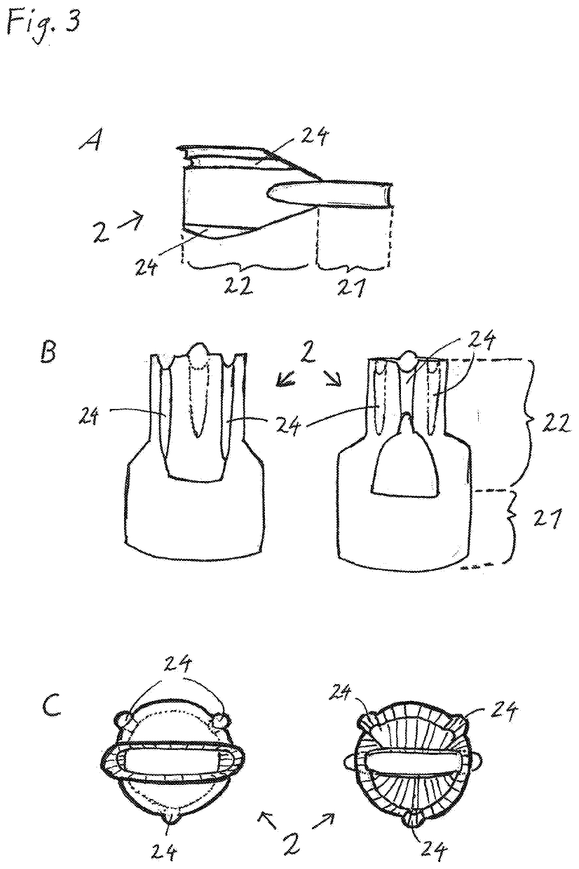

FIG. 2 shows a longitudinal section through a nebulizer unit 1 according to embodiments of the present disclosure with mounted mouthpiece with air channels. The connecting piece 12, on which the mouthpiece 2 is mounted, has a circular cross-section that is complementary to the internal diameter of the mouthpiece. On the underside of the nebulizer unit, a flow channel 104 is attached, which terminates at the rear end with a nozzle, which can be connected to a control unit (not shown here). Its front end, when the mouthpiece 2 is mounted, connects flush with one of the air channels 24 of the mouthpiece 2, resulting in loss-free measurement of the sucked-in air quantity, and therefore of the breath profile. In the embodiment proposed here, the proposed embodiment continues to be advantageously amplified in that the outlet opening of the air channel 24 connected on the flow channel 104 in that portion of the aerosol chamber 120 that is located in the mouthpiece. This forward-shifted pressure take-off point undergoes a greater pressure variation in the course of a respiratory cycle, since the air stream there is faster and thus the dynamic component of the overall pressure is greater or varies more strongly.

FIG. 3 shows, in three partial figures, a five-sided view of the exchangeable mouthpiece 2 of the nebulizer unit 1 according to embodiments of the present disclosure.

Partial figure A is a side view that clearly shows the subdivision of the mouthpiece in the front part 21, which is shaped for gripping with the lips, and the rear part 22, which is shaped appropriately for plugging onto the connecting piece 12 of the nebulizer unit 1. As can be seen here, the latter, by means of bevels shaped at the top and bottom sides, merges continuously into the front portion. Such a continuous transition is advisable for the development of a uniform laminar flow in the interior of the mouthpiece and thus for an, as far as possible complete, removal of the aerosol mist. The lower of the air channels 24 is, at its end facing the nebulizer unit, shaped such that it connects flush to a flow channel 104, that is present thereon, such that a measurement removal point is created for a pressure or flow sensor located in a control unit.

Partial figure B shows the mouthpiece in top view (left partial figure) and bottom view (right partial figure). Of the recesses 24 that, in the condition in which they are plugged onto the mounting piece 12, form the air channels, two are integrally moulded at laterally and one centrally on the underside. The front portion 21 is widened appropriate to the mount. Since the mouthpiece is exchangeable, few compromises need to be made regarding the precise design and size of this part 21, since, in principle, a suitable mouthpiece can be manufactured for each user.

Partial figure C shows the front and rear view (left or right partial figure) of the exchangeable mouthpiece according to embodiments of the present disclosure. It can be clearly seen that the protrusions 24 on the inside thereof, in the condition (taken off from the nebulizer unit) shown here are open so that they can be very easily cleaned. Only when plugged onto the connecting piece 12 is this inside sealed by the outside of the, essentially cylindrical in this preferred embodiment, connecting piece 12, and open air channels 24, in each case only open at the front and rear ends, are formed.

### LIST OF REFERENCE CHARACTERS

1 Nebulizer unit
11 Medication reservoir
12 Connecting piece
120 Aerosol chamber
13 Cap
101 Aerosol generator
102 Sealing ring
103 Retaining structure
104 Flow channel
2 Mouthpiece
21 Front portion
22 Rear portion
24 Air channel, protrusion

Although the embodiments of the present disclosure have been described with reference to preferred embodiments, workers skilled in the art will recognize that changes may be made in form and detail without departing from the spirit and scope of the present disclosure.

1. A nebulizer unit comprising:
a housing;
an aerosol generator, which is supported within the housing by a sealing ring and a retaining structure, and includes an outlet end having an integrally formed mouthpiece connector; and
an exchangeable mouthpiece configured to be received by the mouthpiece connector;
wherein:
one side of the mouthpiece includes at least one air channel, each air channel having a first end that opens into an aerosol chamber; and
the aerosol chamber is delimited at an end face by the aerosol generator and circumferentially by the mouthpiece connector and the mouthpiece.

2. The nebulizer unit according to claim 1, wherein the at least one air channel comprises a plurality of air channels.

3. The nebulizer unit according to claim 1, wherein the at least one air channel consists of three air channels, two of which are integrally formed at one side of the mouthpiece and one is integrally formed on an underside of the mouthpiece.

4. The nebulizer unit according to claim 1, wherein a cross-section of the mouthpiece is cylindrical.

5. The nebulizer unit according to claim 1, wherein the mouthpiece is manufactured from plastic.

6. The nebulizer unit according to claim 1, wherein a second end of each of the at least one air channels is outwardly open.

7. The nebulizer unit according to claim 1, wherein one of the at least one channel is connected to a flow channel of the nebulizer unit.

8. The nebulizer unit according to claim 1, wherein the aerosol chamber includes a portion that forms part of the nebulizer unit and has no openings outwardly.

9. The nebulizer unit according to claim 1, wherein air inlet openings are present in the housing.

10. The nebulizer unit according to claim 1, further comprising a medication reservoir that is closed by a cap.
